(19) United States
(12) Patent Application Publication
(10) Pub. No.: US 2018/0289619 A1
(43) Pub. Date: Oct. 11, 2018

(54) GLUCOSE STRIP SYSTEM

(71) Applicant: Mark Culleton, Billerica, MA (US)

(72) Inventor: Mark Culleton, Billerica, MA (US)

(21) Appl. No.: 15/480,779

(22) Filed: Apr. 6, 2017

(51) Int. Cl. A61K 9/00; A61K 31/7004; A61K 9/70; A61J 1/00; B65D 43/16; B65D 83/08

(52) U.S. Cl. CPC A61K 9/0056; A61K 31/7004; B65D 83/0805; A61J 1/00; B65D 43/16; A61K 9/7007

(57) ABSTRACT

A glucose strip system which includes a container having a top surface, a bottom surface, and an inner volume, and at least one glucose strip having at least one flavoring agent. The glucose strip system includes the container and the at least one glucose strip. The container has the top surface, the bottom surface, and the inner volume. The bottom surface is substantially planar allowing at least one glucose strip able to lay flat within the inner volume between the top surface and the bottom surface of the container. At least one glucose strip is able to be orally administered by a user for regulating, sustaining, and increasing blood sugar levels of the user.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of systems and compositions to regulate blood sugar level of existing art and more specifically relates to a glucose strip that increases blood sugar level instantly by oral administration.

### RELATED ART

Low blood sugar or Hypoglycemia, is a medical condition that affects several people. Hypoglycemia exists when blood sugar levels are, or become, lower than normal (typically below 70 mg/dL, and sometimes below 50 mg/dL). Hypoglycemia may occur when plasma insulin levels rise to levels that are higher than normal resulting in blood glucose levels dropping to levels that are insufficient to provide enough energy for normal bodily functions. Hypoglycemia can be exacerbated by exercise or other activities which require higher levels of blood glucose. Hypoglycemia is a well-studied side effect of medications used in diabetes treatment, but can also be a side-effect of other medications, or result from other diseases, hormone and enzyme deficiencies, and tumors. Hypoglycemia can also be brought on by exacerbating circumstances in an otherwise healthy individual.

Treatments for hypoglycemia are well established. A common technique is to ingest some form of sugar, such as fruit juice, glucose tablets, milk, sugar, honey, or hard candy, followed by waiting for 15 minutes to see if symptoms abate or blood glucose levels rise to an acceptable level. If symptoms persist or blood glucose levels continue to be below acceptable levels, the individual repeats ingestion of the sugar containing substance and waits again to see the effect on blood sugar level. A suitable, safe, and efficient solution is desired to regulate, stabilize and increase blood sugar.

U.S. Pat. No. 8,609,165 to Kevin Andrews relates to a dietary supplement for optimizing glucose levels, increasing energy, and improving general health. The described dietary supplement for optimizing glucose levels, increasing energy, and improving general health includes a dietary supplement which can optimize glucose levels, increase energy, and provide other health benefits including combinations of alpha lipoic acid, vanadium, zinc, vitamin C and biotin.

### SUMMARY OF THE INVENTION

In view of the foregoing disadvantages inherent in the known compositions to regulate blood sugar levels art, the present disclosure provides a novel glucose strip system. The general purpose of the present disclosure, which will be described subsequently in greater detail, is to provide a glucose strip system.

A glucose strip system is disclosed herein. The glucose strip system includes a container having a top surface, a bottom surface, and an inner volume, and at least one glucose strip having at least one flavoring agent. The glucose strip system includes the container and the at least one glucose strip in functional relation to each other. The container is defined by the top surface, the bottom surface, and the inner volume. The bottom surface is substantially planar allowing at least one glucose strip able to lay flat within the inner volume between the top surface and the bottom surface of the container. At least one glucose strip is able to be orally administered once removed by a user for regulating, sustaining, and increasing blood sugar levels of the user. The present invention includes a composition that can be placed directly on the soft pallet of a tongue and absorbed directly into the blood stream. The present invention is designed as such to provide an improved and new method and composition of treating hypoglycemia by glucose strips.

The present invention provides an improved method to treat low blood sugar level. As such, the general purpose of the present invention, which will be described subsequently in greater detail, is to provide a new and improved method to increase or normalize blood sugar. The container is a ‘pull out’ container and small in size so that it may be carried in pocket or purse or fanny pack, etc for convenient use. The glucose strips are administrated orally by placing the strip on a tongue of the user directly to regulate blood sugar levels.

The glucose strips may comprise a dissolvable thin oral film made of water, sugar, glycerin and at least one flavoring agent. The glucose strips are designed to be absorbed through the soft pallets of the tongue to be absorbed in to the blood stream. The present invention provides a system and composition that eliminates the need to chew or swallow the candy or tablet to regulate blood sugar.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a glucose strip system, constructed and operative according to the teachings of the present disclosure.

FIG. 1 is a perspective view of the glucose strip system during an ‘in-use’ condition, according to an embodiment of the disclosure.

FIG. 2 is a perspective view of the glucose strip system of FIG. 1, according to an embodiment of the present disclosure.

FIG. 3 is a perspective view of the glucose strip system of FIG. 1, according to an embodiment of the present disclosure.

FIG. 4 is a perspective view of the glucose strip system of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a composition to regulate blood sugar level and more particularly to a glucose strip system which provides a system and composition to increase blood sugar level by oral administration. The composition may be placed directly on the soft pellets of tongue and absorbed directly into the blood stream as an efficient and effective means to regulate blood sugars.

Generally, glucose strip system provides provide a method to increase or normalize blood sugar. The present invention treats hypoglycemia by glucose strips and provides a glucose strip dispenser that comprises; sugar strips and a container. The glucose strips are administrated orally by putting the strip directly on a tongue. The glucose strips regulate blood sugar level by providing glucose strips that are made of a dissolvable thin oral film and comprise water, sugar, glycerine and flavoring agent(s). The glucose strips may be absorbed through the tongue to be absorbed into the blood stream quickly for maximum efficiency. The glucose strip system provides an effective system and composition that eliminates the need to chew or swallow the candy or tablet to regulate blood sugar.

The dispenser contains glucose strips and container. The container is preferably made of plastic. The container has a top surface that is a pull out surface. This pull out surface is an open enclosure for the glucose strips. The top surface is pulled out to open the container and get a strip out from the container. The bottom surface of the container is flat. The glucose strips are laid flat in the container. The glucose strips are made of thin film that is coated with water soluble sugar. The thin film further comprises water, glycerine and flavoring agent.

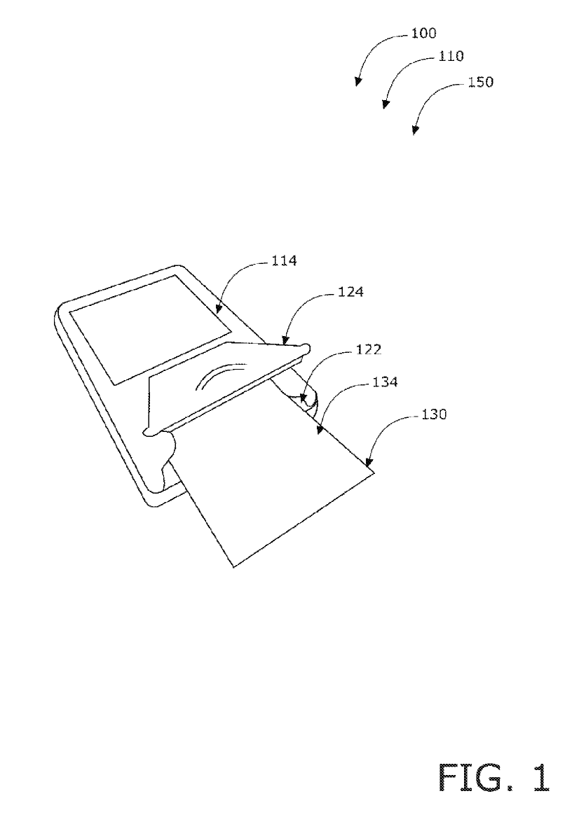

Referring now more specifically to the drawings by numerals of reference, there is shown in FIGS. 1-4, various views of a glucose strip system 100. FIG. 1 shows a glucose strip system 100 during an ‘in-use’ condition 50, according to an embodiment of the present disclosure. Here, the glucose strip system 100 may be beneficial for use by a user 40 to increase blood sugar level by oral administration. As illustrated, the glucose strip system 100 may include a container 110 having a top surface 114, a bottom surface 118, and an inner volume 122, and at least one glucose strip 130 having at least one flavoring agent 134. The container 110 contains a fix number of glucose strips. In a preferred embodiment, each container preferably contains 15 glucose strips 130. At least one glucose strip 130 may be placed directly on the tongue to be dissolved directly in saliva so that it may be absorbed in to the blood stream in quick fashion.

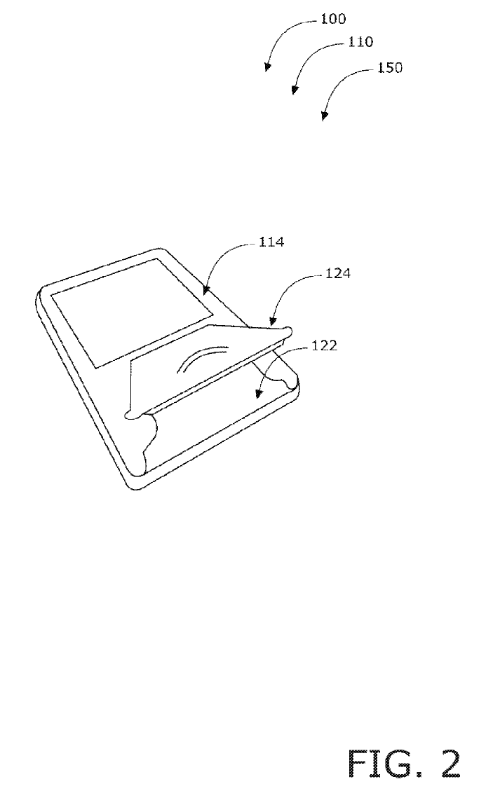

FIG. 2 shows a perspective view of the glucose strip system of FIG. 1, according to an embodiment of the present disclosure. As above, the glucose strip system 100 may include a container having a flat bottom surface 118 and looks triangular in shape when closed. An opening member 124 of the top surface 114 of the container 110 is slightly higher than the opposing end. This provides an easy method retrieve at least one glucose strip 130 out from the container 110. The glucose strip system 100 comprises at least one glucose strip 130 and the container 110 that holds at least one glucose strip 130 within its inner volume 122. The container 110 is preferably made of plastic and small in size. At least one glucose strip 130 made of easily dissolvable thin oral film; the film may be coated with water, sugar, glycerine and at least one flavoring agent 134. In a preferred embodiment, the container contains 15 of the at least one glucose strips 130 in it.

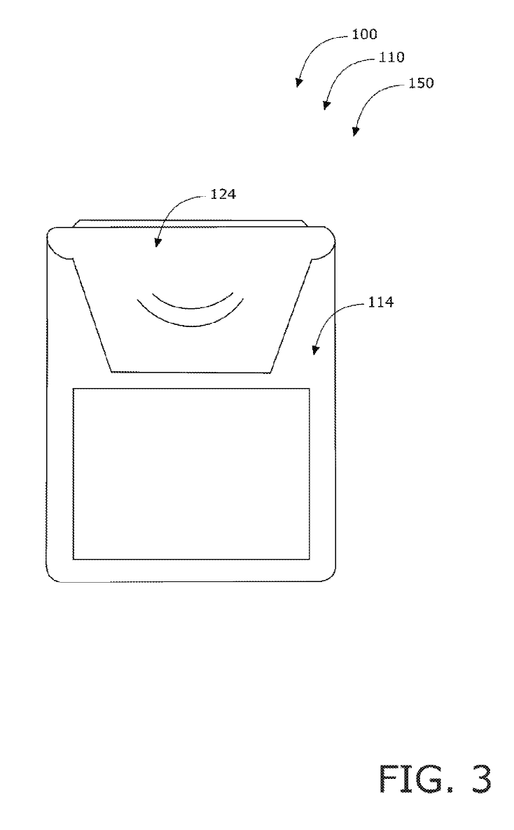

FIG. 3 is a perspective view of the glucose strip system of FIG. 1, according to an embodiment of the present disclosure. In a preferred embodiment, at least one glucose strip 130 comprises a roll of water soluble sugar thin film. Further, the film is coated with a measured ‘dose’ of sugar that is that is dissolvable in saliva. For example, 15 grams of glucose is coated per strip with 40% of rapidly dissolving sugar preferably dextrose. The film is coated with purified water, measured amount of sugar, glycerin and at least one flavoring agent 134.

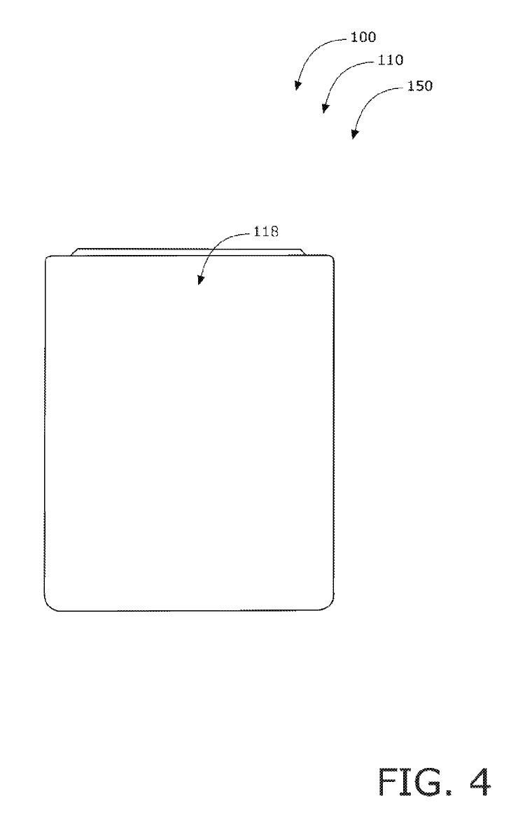

FIG. 4 is a perspective view of the glucose strip system of FIG. 1, according to an embodiment of the present disclosure. The present invention includes a system and composition for the treatment of hypoglycemia or low blood sugar level. The present invention provides that the glucose strip system 100 may include a container and at least one glucose strip 130 to work in functional combination. The container 110 is a small size plastic carrying device that can be placed in a pocket or the like. At least one glucose strip 130 is made of purified water, easily dissolvable sugar, glycerin and flavoring agent. At least one glucose strip 130 may be directly placed on the tongue to regulate blood sugar levels.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A glucose strip system, the system comprising:
a container having
a top surface,
a bottom surface, and
an inner volume, and
at least one glucose strip with
at least one flavoring agent;
wherein said glucose strip system comprises said container and said at least one glucose strip;
wherein said container comprises said top surface, said bottom surface, and said inner volume;
wherein said bottom surface is substantially planar, said at least one glucose strip able to lay flat within said inner volume between said top surface and said bottom surface of said container;
wherein said at least one glucose strip comprises at least one flavoring agent; and
wherein said at least one glucose strip able to be orally administered by a user for regulating, sustaining, and increasing blood sugar levels of the user.

2. The glucose strip system of claim 1, wherein said container comprises an opening member for removing said at least one glucose strip.

3. The glucose strip system of claim 1, wherein said container comprises plastic.

4. The glucose strip system of claim 1, wherein said at least one glucose strip is coated with water, sugar, glycerine, and said at least one flavoring agent.

5. The glucose strip system of claim 1, wherein said at least one glucose strip is dissolvable in saliva.

6. The glucose strip system of claim 1, wherein said at least one glucose strip comprises 15 grams of glucose coated with 40% of rapidly dissolving sugar.

7. The glucose strip system of claim 1, wherein said sugar comprises dextrose.

8. The glucose strip system of claim 1, wherein said sugar comprises monosaccharide.

9. The glucose strip system of claim 1, wherein said at least one glucose strip comprises sugar which is absorbed through soft pallets of a tongue to be absorbed in a blood stream of the user.

10. The glucose strip system of claim 1, wherein said container is slightly wider at near said opening member providing ease of removal of at least one glucose strip through said opening member.

11. The glucose strip system of claim 1, wherein said opening member is hingedly coupled to said top surface said container.

12. The glucose strip system of claim 1, wherein said inner volume of said container houses approximately 15 of said at least one glucose strips.

13. The glucose strip system of claim 1, wherein said at least one glucose strip comprises a dissolvable thin oral film.

14. The glucose strip system of claim 1, wherein said at least one glucose strip is orally administered.

15. A glucose strip system, the system comprising:
a container having
a top surface,
a bottom surface, and
an inner volume, and
at least one glucose strip;
at least one flavoring agent;
wherein said glucose strip system comprises said container and said at least one glucose strip;
wherein said container comprises said top surface, said bottom surface, and said inner volume;
wherein said container comprises an opening member for removing said at least one glucose strip;
wherein said opening member is hingedly coupled to said top surface said container;
wherein said container comprises plastic;
wherein said bottom surface is substantially planar, said at least one glucose strip able to lay flat within said inner volume between said top surface and said bottom surface of said container;
wherein said container is slightly wider at near said opening member providing ease of removal of at least one glucose strip through said opening member;
wherein said inner volume of said container houses approximately 15 of said at least one glucose strips;
wherein said at least one glucose strip comprises at least one flavoring agent;
wherein said at least one glucose strip is coated with water, sugar, glycerine, and a at least one flavoring agent;
wherein said at least one glucose strip comprises a dissolvable thin oral film;
wherein said at least one glucose strip is dissolvable in saliva;
wherein said at least one glucose strip comprises 15 grams of glucose coated with 40% of rapidly dissolving sugar;
wherein said sugar comprises dextrose;
wherein said at least one glucose strip comprises sugar which is absorbed through soft pallets of the tongue to be absorbed in a blood stream of the user; and
wherein said at least one glucose strip able to be orally administered by a user for regulating, sustaining, and increasing blood sugar levels of the user.
